(19) United States
(12) Patent Application Publication
(10) Pub. No.: US 2012/0255556 A1
(43) Pub. Date: Oct. 11, 2012

(54) FALLOPIAN TUBE FILTER

(76) Inventor: Justin D. Renfrow, Bloomington, IN (US)

(21) Appl. No.: 13/438,153

(22) Filed: Apr. 3, 2012

Related U.S. Application Data
(60) Provisional application No. 61/472,064, filed on Apr. 5, 2011.

(51) Int. Cl. A61F 6/22

(52) U.S. Cl. 128/831

(57) ABSTRACT

Apparatuses and methods are provided for a removable, non-permanent indwelling contraceptive fallopian tube filter that provides an immediate, customized anatomically-fit barrier between sperm and egg to prevent pregnancy without the use of drugs, hormones, or altering the anatomy.

### CROSS-REFERENCE TO RELATED APPLICATIONS

This application claims priority under to U.S. Provisional Application No. 61/472,064, filed on Apr. 5, 2011, the entirety of which is hereby fully incorporated by reference herein.

### TECHNICAL FIELD

The present invention relates to indwelling medical devices and more specifically, contraceptive devices used to prevent pregnancy.

### BACKGROUND

Medicinal contraceptive means used to prevent pregnancy, such as those commonly known as “the pill,” are generally reversible such that when no longer used, the patient can become pregnant. However, such contraceptive means suffer from a variety of shortcomings. For example, use of drugs or hormones to suppress a female's production of natural hormones to prevent release of an egg or increase vaginal discharge to prevent sperm from reaching the egg, subject the patient to drugs and/or hormones that she may be unwilling or unable to take. Additionally such means often require periodic trips to the doctor or periodic use, for example, by taking a pill at the same time daily, or the effectiveness diminishes. Often times, the patient may forget to timely take the pill, thus resulting in pregnancy.

Use of condoms as a contraceptive means provide several shortcomings. For example, condoms have to be readily available at the time of intercourse, are viewed by some to as being uncomfortable or an annoyance, and even if properly used, may break resulting in pregnancy.

Indwelling contraceptive devices are devices implanted into a woman that prevent pregnancy either by release of a drug or hormone, or by altering a woman's reproductive anatomy. Such devices are generally touted as being beneficial because there are no pills to remember to take and no devices to use during intercourse. However, indwelling contraceptive devices suffer from several shortcomings as contraceptive means.

For example, Essure® (Conceptus, Inc., Mountain View, Calif.) provides permanent, non-reversible, indwelling contraception by placing a micro-insert into each of the fallopian tubes, whereby the body, over a period of time, grows tissue around the micro-inserts, thus blocking the fallopian tubes to prevent sperm from reaching and fertilizing an egg. Occlusion of the fallopian tubes is not immediate; instead, it normally takes a period of about three-months for a sufficient amount of tissue to occlude the fallopian tubes.

Adiana® (Hologic, Inc., Bedford, Mass.) provides permanent, non-reversible, indwelling contraception. A delivery catheter is passed through the vagina and cervix and up to the fallopian tubes. Radiofrequency energy is then emitted to create a lesion into each of the fallopian tubes. The Adiana® insert, which is about the size of a grain of rice, is then inserted into each of the fallopian tubes at the lesion site. Over a period of about three months, tissue grows in and around the inserts eventually occluding each of the fallopian tubes to prevent sperm from reaching and fertilizing an egg.

While Essure® and Adiana® avoid having the patient remember to take a pill or use barrier means, such as a condom, to prevent pregnancy, the fact that such contraception means are permanent, non-reversible, and alter one's anatomy is a drawback. Moreover, even the known permanent contraceptive means take time to become effective due to the change in female anatomy required to create an effective barrier necessitating the use of a back-up contraceptive means during the interim tissue-growth period.

Merina® (Bayer HealthCare Pharmaceuticals, Berkeley, Calif.) is an indwelling contraception device placed into the uterus that is non-permanent and reversible. It prevents pregnancy by emitting the hormone levonorgestrel directly into the uterus. Although Merina is not permanent, the fact that it emits a hormone is a drawback.

### BRIEF SUMMARY

In a first aspect, a contraceptive device is provided having a plurality of longitudinal wires each including a body portion and a securing portion, each wire disposed within a plane through a longitudinal axis of the contraceptive device, wherein the body portion of adjacent wires are connected to each other at a plurality of locations by a plurality of circumferentially disposed wires; wherein the securing portion of the wires are unconnected and include a uniform cross-sectional area and are configured to curve outward from the longitudinal axis; further wherein the securing portion is atraumatic and configured to engage and penetrate tissue and resist tissue in-growth thereabout to permit removal of an entirety of the contraceptive device with minimal tissue damage; wherein the body portion and the securing portion are configured to move between a radially expanded state and an unexpanded state, wherein an outer diameter of the securing portion when in the radially expanded state is greater than an outer diameter of the securing portion when in the unexpanded state; a retrieval member connected to the body portion; and a film in communication with at least a portion of the body portion configured to seal the area between adjacent wires from the passage of sperm and eggs.

In a second aspect, a method of delivering a fallopian tube filter is provided, the method including providing a fallopian tube filter including a retrieval member wherein the retrieval member includes an engagement portion configured to engage a grasping member for the removal of the fallopian tube filter, wherein the fallopian tube filter is configured for reversibly sealing a passageway from sperm and eggs and includes an atraumatic profile, wherein the fallopian tube filter is configured to engage and penetrate, tissue and resist tissue in-growth thereabout to permit removal of an entirety of the fallopian tube filter with minimal tissue damage; compressing the fallopian tube filter; directing the fallopian tube filter to a proximal fallopian tube as deep as the tubal isthmus; and deploying the fallopian tube filter at the proximal fallopian tube as deep as the tubal isthmus, wherein the retrieval member extends towards a uterus cavity, and wherein the fallopian tube filter expands and applies a radial force to an interior surface of the fallopian tube reversibly sealing the fallopian tube from the passage of sperm and eggs.

In a third aspect, an occlusion medical device is provided having a conical configuration of a plurality of atraumatic wires including a body portion and a securing portion, wherein the securing portion is configured to move between a radially expanded state and an unexpanded state, wherein an outer diameter of the securing portion when in the radially expanded state is greater than the outer diameter of the securing portion when in the unexpanded state, further wherein the securing portion is atraumatic and configured to engage and penetrate tissue and resist tissue in-growth thereabout to permit removal of an entirety of the medical device with minimal tissue damage; a retrieval member connected to the body portion; and a sealing member in communication with at least a portion of the body portion to seal the medical device from the passage of a fluid and a solid therethrough.

### BRIEF DESCRIPTION OF THE SEVERAL VIEWS OF THE DRAWINGS

The embodiments will be further described in connection with the attached drawing figures. It is intended that the drawings included as a part of this specification be illustrative of the exemplary embodiments and should in no way be considered as a limitation on the scope of the invention. Indeed, the present disclosure specifically contemplates other embodiments not illustrated but intended to be included in the claims.

FIG. 1A illustrates a side view of an illustrative fallopian tube filter in an expanded state;

FIG. 1B illustrates a side view of an illustrative fallopian tube filter in an unexpanded, compressed state;

FIG. 1C illustrates a side view of an illustrative fallopian tube filter having an alternate retrieval member;

FIG. 1D illustrates a side view of an illustrative fallopian tube filter having an alternate retrieval member;

FIG. 2 illustrates the device, illustrated FIG. 1A, in use;

FIG. 3 illustrates a system for deployment of an illustrative fallopian tube filter;

FIG. 4 illustrates a system for removal of an illustrative fallopian tube filter; and

FIG. 5 illustrates a method for placing an illustrative fallopian tube filter to prevent pregnancy.

### DETAILED DESCRIPTION OF PRESENTLY PREFERRED EMBODIMENTS

The exemplary embodiments illustrated herein provide exemplary removable contraceptive apparatuses and methods for occluding fallopian tubes to prevent sperm from reaching an egg and thereby prevent pregnancy.

The present invention is not limited to those embodiments described herein, but rather, the disclosure includes all equivalents and those intended to be included in the claims.

Moreover, the embodiments and principles illustrated can be used and applied to any portion of the body benefiting from an indwelling medical device that occludes a lumen from the passage of fluid or solid matter and are not limited to the sizes, shapes, or configurations illustrated herein.

Throughout, patient is not limited to a human being; animals and other living organisms are contemplated. User is contemplated throughout the disclosure as being anyone or thing capable of using the device or performing the methods, including but not limited to, a human being and machine.

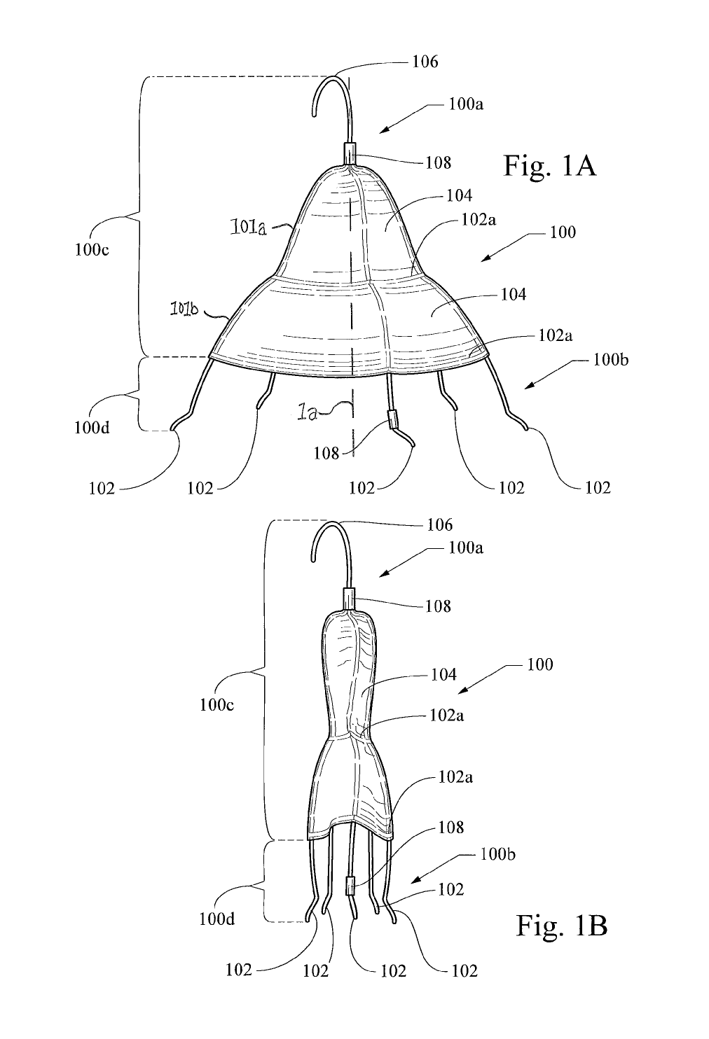

A more detailed description of the embodiments will now be given with reference to FIGS. 1A-5. Throughout the disclosure, like reference numerals and letters refer to like elements. The present disclosure is not limited to the embodiments illustrated; to the contrary, the present disclosure specifically contemplates other embodiments not illustrated but intended to be included in the claims.

It has been discovered that the creation of an indwelling, non-permanent, reversible contraceptive device, including but not limited to, a fallopian tube filter, can be configured such that it prevents pregnancy without the use of drugs, hormones, or altering the patient's anatomy, that is effective immediately without requiring tissue in-growth to secure the device, and that is completely removable.

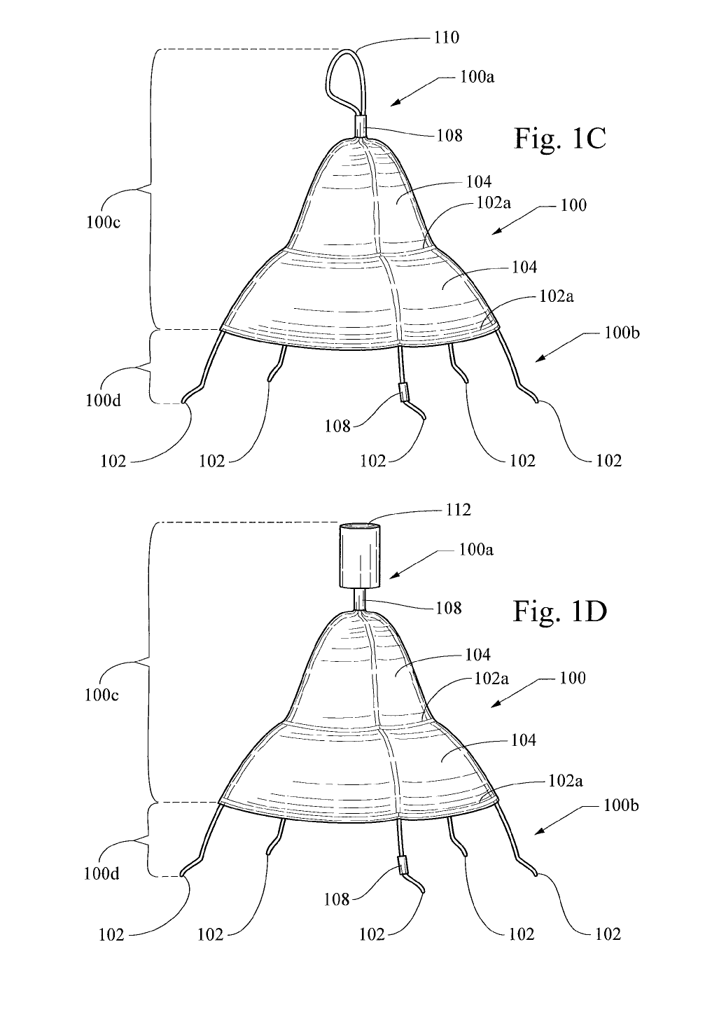

FIG. 1A illustrates a side view of contraceptive fallopian tube filter 100 in a radially expanded state having proximal portion 100a, distal portion 100b, and being configured into a conical-like-shaped umbrella from a plurality of flexible longitudinal wires 102 such that the outer diameter of proximal portion 100a of fallopian tube filter 100 is less than outer diameter of distal portion 100b of fallopian tube filter 100 when in an expanded state. Wires 102 are atraumatic and are configured to cleanly penetrate tissue at their tips. The wires 102 are configured to position themselves and the remainder of the filter 100 with respect to the fallopian tube tissue to minimize tissue ingrowth therein, which in combination of the relatively clean penetration of the wires 102 into the tissue minimizes the damage or trauma to tissue if the filter 100 is removed. FIG. 2 illustrates fallopian tube filter 100 in use.

Referring to FIGS. 1A and 2, fallopian tube filter 100 is an indwelling contraceptive device that is deployed in the proximal fallopian tube as deep as the tubal isthmus, wherein once placed, retrieval member hook 106 extends out from fallopian tube and into the cavity of the uterus (as illustrated in FIG. 2) to provide a means for retrieval of fallopian tube filter 100 should contraception no longer be needed or desired.

As illustrated, the plurality of longitudinal wires 102 each have body portion 100c and securing portion 100d, disposed within a plane disposed through a longitudinal axis 1 of fallopian tube filter 100, such that body portion 100c of the wires 102 are connected to each other at a plurality of locations by a plurality of circumferentially disposed wires 102a. Optionally, longitudinal wires 102 need not have circumferentially disposed wire connections; instead, longitudinal wires 102 may optionally be connected in the proximal-most portion. Other configurations are contemplated.

In some embodiments shown on FIG. 1A, the body portion 100c includes a distal portion 101b and a proximal portion 101a. The distal and proximal portions 101b, 101a are formed from the same wires 102, with the wires 102 formed to include a discontinuity in curvature to differentiate between the two portions. In some embodiments, the proximal portion 101a is formed with a substantially conical portion with a linearly increasing diameter along the longitudinal axis 1a. Other shapes of the proximal portion 101a are contemplated. The distal portion 101b is formed with an increasing diameter that increases at a faster rate than the proximal portion 101a, which may increase at a non-linearly increasing rate. The relatively larger distal portion 101b is normally sized with a larger diameter (along its length) than the nominal diameter of a fallopian tube to provide strong fixation thereto. In some embodiments, the proximal portion 101b has an end diameter also larger than the nominal diameter of a fallopian tube such that the proximal portion (as surrounded by the film layer 104, discussed below) blocks the fallopian tube.

Securing portion 100d of the wires 102 are unconnected and each may include a uniform cross-sectional area and may be configured to curve outward from the longitudinal axis 1 of fallopian tube filter 100. Securing portion 100d is atraumatic and configured to engage and penetrate tissue while being constructed and positioned to resist tissue in-growth thereabout to permit removal of fallopian tube filter 100 with minimal tissue damage. It is realized that while some damage may occur, such as a scraping of the cells, the damage to the tissue site, if any, is minimal and will not result in significant bleeding or other anatomical destruction or scarring to the patient. Accordingly, reversal of the contraceptive procedure is easy and presents a reduced risk to the patient. Moreover, because the entirety of fallopian tube filter 100 is removable, no foreign structure remains dwelling within the patient after the procedure is reversed.

Wires 102 and 102a may be made from a nickel titanium alloy (nitinol) although other materials are contemplated that are elastic, including those that are superelastic such that the material is configurable into a biased position, and when deformed from that position will tend to resume the initial biased position. Other materials that are contemplated are stainless steel or other medical grade material exhibiting this elasticity. Wires 102, 102a are heat set to assume an open, uncompressed, expanded state, as illustrated in FIG. 1A, and are able to move between a wholly or partially closed, compressed, unexpanded state, as illustrated in FIG. 1B, when a sufficient force (whether a point force or a force applied over some or all of the surface area) is applied to the outer surface of wires 102, 102a, including but not limited to, during deployment and retrieval, and thereafter return to the biased open position (as illustrated in FIG. 1A) when the force is removed.

Body portion 100c is coated with a film layer 104. Film 104 is preferably Thoralon® (Thoratec Corp., Pleasanton, Calif.), a polyetherurethane urea blended with a siloxane containing surface modifying additive providing an effective sealing member means covering body portion 100c so as to seal fallopian tube filter 100 and prevent sperm and eggs from passing therethrough. It is contemplated that film 104 can be applied to the outer surface and/or the inner surface of wires 102, 102a such that the area between adjacent wires 102, 102a will be sealed to provide a barrier from eggs and sperm traveling therethrough.

Other film coatings are contemplated, including but not limited to, silicone, depending upon the needs of the patient, provided that the film coating prevents the passage of sperm and eggs. Indeed it is also contemplated that fallopian tube filter 100 may be coated in whole or part with a material or drug to prevent tissue in-growth, bacterial growth, or to kill sperm; it is also contemplated that fallopian tube filter 100 may be coated in whole or in part with drugs, including but not limited to, drugs that would elute from fallopian tube filter 100 thereby providing a therapeutic treatment or address another patient need.

Fallopian tube filter 100 optionally includes any number of markers 108 that provide for direct endoscopic visualization to aid in the placing or retrieving of fallopian tube filter 100. However, it is contemplated that markers can also provide for indirect visualization, including but not limited to, for example, through the use of ultrasound. As illustrated, markers 108 are located on proximal portion 100a and distal portion 100b of fallopian tube filter 100; however, it is contemplated that markers can be located elsewhere in any number, including none.

It is contemplated that markers may also be made in whole or in part from materials, including but not limited to, stainless steel, radiopaque materials, Platinum-Iridium alloy or echogenic materials, including but not limited to, gold and tungsten having surface irregularities. An echogenic material includes surface irregularities that reflect ultrasonic waves and thus, allow the material to be seen with ultrasonic imaging devices. Echogenic techniques are described in U.S. Pat. No. 5,081,997 and U.S. Pat. No. 5,289,831 and are hereby incorporated by reference in their entirety. Thus, markers 108 provide for visualization outside the patient using a direct or indirect visualization device, including but not limited to, hysteroscopy, fluoroscopy, x-ray, direct endoscopic visualization, ultrasound, or magnetic resonance imaging (MRI).

FIG. 1B illustrates a side view of fallopian tube filter 100 in a compressed, unexpanded state. Wires 102 are illustrated as being disposed adjacent to each other and collectively in a closed position such that fallopian tube filter 100 is able to be deployed into fallopian tube or retrieved therefrom.

Wires 102 of fallopian tube filter 100 are about 2-3 cm long although other sizes, dimensions, and configurations are contemplated depending upon the needs of the patient. Wires 102, are preferably blunt and potentially curved or arcuate at distal portion 100b and have a smooth outer surface so as to prevent trauma to the anatomy, although other configurations are contemplated.

Hook 106 is about 2 cm long although other dimensions and configurations are contemplated depending upon the needs of the patient.

When in an expanded state (illustrated in FIG. 1A), distal portion 100b of fallopian tube filter 100 has an outer diameter of about 3 Fr (⅓ mm). When in a partial or fully compressed state (illustrated in FIG. 1B), fallopian tube filter 100 has an outer diameter less than its outer diameter when in an uncompressed, expanded state (illustrated in FIG. 1A).

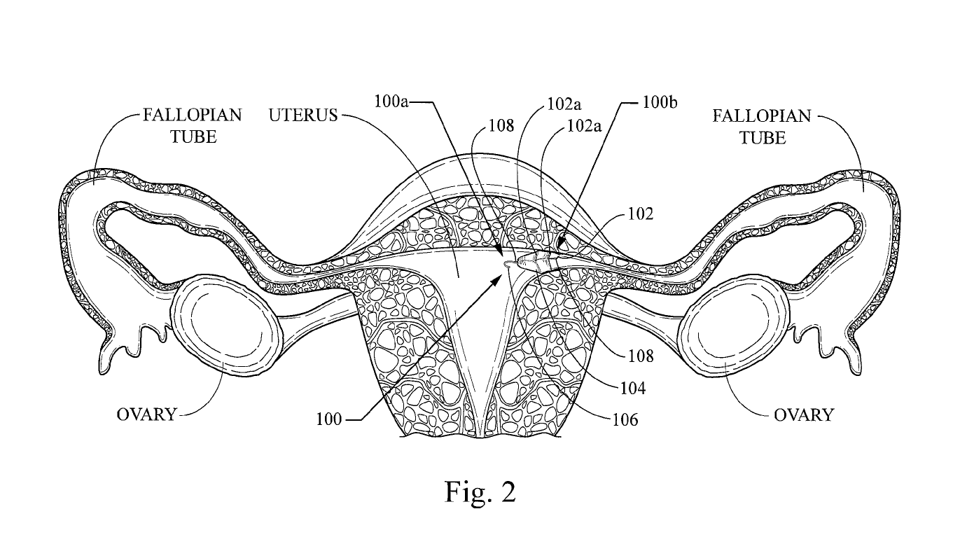

FIG. 3 illustrates a system for deployment of the compressed fallopian tube filter 100 (illustrated in FIG. 1B). Referring to FIGS. 2 and 3, fallopian tube filter 100 is deployed, for example, using a deployment system having a working channel of a hysteroscope 202 and pusher 204. Hysteroscope 202 is directed to the proximal fallopian tube as deep as the tubal isthmus. Pusher 204 pushes fallopian tube filter 100 out from working channel of hysteroscope 202 and into fallopian tube 100 such that proximal portion 100a of fallopian tube filter 100 having retrieval hook 106 extends proximally into or towards uterus cavity. As fallopian tube filter 100 moves out from working channel of hysteroscope 202 and into fallopian tube, wires 102 of fallopian tube filter 100 begin to assume an expanded state (as illustrated in FIG. 1A) and apply a radial force to the inner surface of fallopian tube (as illustrated in FIG. 2) such that fallopian tube filter 100 remains in place. The outwardly directed radially force is the result of configuring the outer diameter of fallopian tube filter 100 to be somewhat larger than the inner diameter of fallopian tube, thereby providing an instant, customized anatomically-fit barrier between egg and sperm without altering the female anatomy or using drugs or hormones.

Use of other delivery means is contemplated, including but not limited to, the use of a catheter for vaginal-directed delivery or delivery percutaneously. Indeed it is also contemplated that fallopian tube filter 100 can be directed for placement or removal using a visualization means, including but not limited to, hysteroscopy, fluoroscopy, x-ray, direct endoscopic visualization, ultrasound, or MRI.

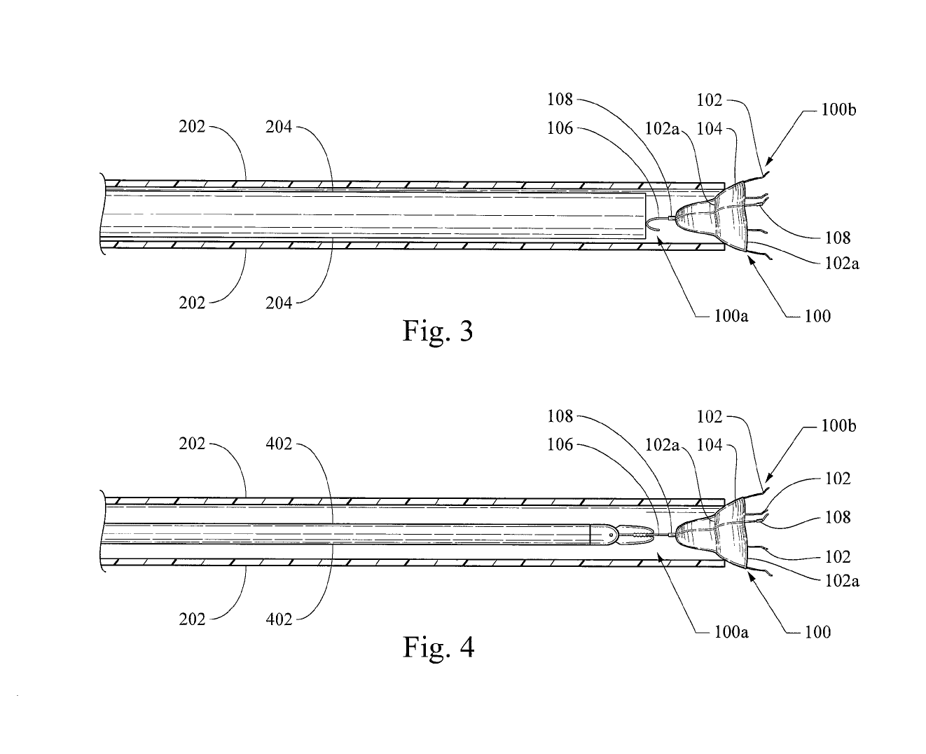

FIG. 4 illustrates a system for removal of fallopian tube filter 100. Referring to FIGS. 2 and 4, fallopian tube filter 100 is retrievable from the patient using a retrieval member means, including but not limited to, hook 106 at proximal portion 100a of fallopian tube filter 100. Hook 106 provides a point of engagement at which grasping member 402 or other tool, including but not limited to, a hook, loop, and magnet, engages fallopian tube filter 100 and removes it when pulled proximally out from fallopian tube. Other retrieval member means are contemplated, including but not limited to, loop 110 (illustrated in FIG. 1C) and magnet 112 (illustrated in FIG. 1D, whereby the grasping member would include an opposite polarity magnet to engage magnet 112). As fallopian tube filter 100 is pulled in a proximal direction, it compresses and reduces its outer diameter to be less than the inner diameter of fallopian tube, such that fallopian tube filter 100 is retrievable such that the entirety of fallopian tube filter 100 is removable.

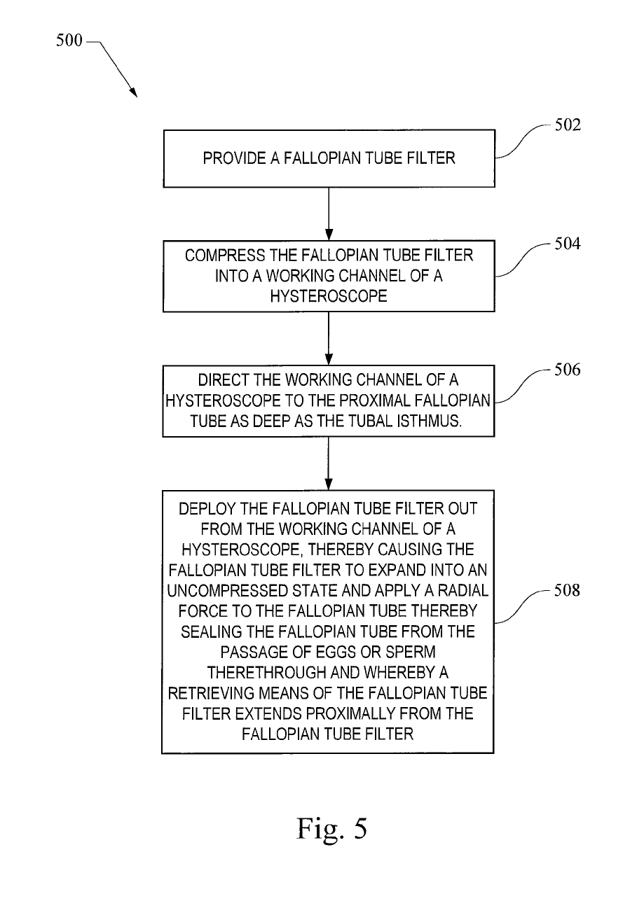

FIG. 5 illustrates a method for placing a fallopian tube filter to prevent pregnancy 500. At block 502, a fallopian tube filter is provided, including but not limited to, that illustrated in FIGS. 1A, 1C, and 1D.

At block 504, the fallopian tube filter is compressed into a working channel of a hysteroscope or other delivery means, including but not limited to, a catheter.

At block 506, the working channel of the hysteroscope (or other delivery system means) is directed to the proximal fallopian tube as deep as the tubal isthmus.

At block 508, the fallopian tube filter is deployed out from the working channel of the hysteroscope (or other delivery system means), thereby causing the fallopian tube filter to expand into an uncompressed state and apply a radial force to the interior surface of fallopian tube thereby sealing the fallopian tube from the passage of eggs or sperm therethrough and whereby a retrieval member means of the fallopian tube filter, including but not limited to, that illustrated in FIGS. 1A, 1C, and 1D, extends proximally from the fallopian tube filter, including but not limited to, into the uterus cavity or near the proximal fallopian tube as deep as the tubal isthmus.

The method can be repeated to provide a barrier in the other fallopian tube if necessary to prevent pregnancy.

The fallopian tube filter can be retrieved by directing a grasping device, including but not limited to, that illustrated in FIG. 4, through the working channel of the hysteroscopy (or other retrieval system means, including but not limited to, a catheter), engaging the retrieval member means of the fallopian tube filter, including but not limited to, that illustrated in FIGS. 1A, 1C, and 1D, and pulling it proximally such that fallopian tube filter compresses as it is removed.

Indeed, it is contemplated that a visualization device can be utilized during deployment and/or retrieval of the fallopian tube filter.

From the foregoing, it can be seen that the present disclosure illustrates apparatuses and methods for a removable, non-permanent indwelling contraceptive device, such as a fallopian tube filter, that provide an immediate, customized anatomically-fit barrier between sperm and egg to prevent pregnancy without the use of drugs, hormones, or altering the anatomy.

What is claimed is:

1. A contraceptive device comprising:
a plurality of longitudinal wires each comprising a body portion and a securing portion disposed within a plane through a longitudinal axis of the contraceptive device, wherein the body portion of adjacent wires are connected to each other at a plurality of locations by a plurality of circumferentially disposed wires;
wherein the securing portion of the wires are unconnected and each comprise a uniform cross-section and are configured to curve outward from the longitudinal axis;
further wherein the securing portion is atraumatic and configured to engage and penetrate tissue and resist tissue in-growth thereabout to permit removal of an entirety of the contraceptive device with minimal tissue damage;
wherein the body portion and the securing portion are configured to move between a radially expanded state and an unexpanded state, wherein an outer diameter of the securing portion when in the radially expanded state is greater than an outer diameter of the securing portion when in the unexpanded state;
a retrieval member connected to the body portion; and
a film in communication with at least a portion of the body portion configured to seal the area between adjacent wires from the passage of sperm and eggs.

2. The contraceptive device of claim 1, wherein the film comprises Thoralon.

3. The contraceptive device of claim 1, wherein body portion comprises a proximal section and a distal section disposed distally of the proximal section, wherein a diameter of the body portion increases distally along the longitudinal axis, and the diameter of the distal section increases at a faster rate than the diameter of the proximal section.

4. The contraceptive device of claim 1, wherein at least a portion of the plurality of longitudinal wires or plurality of circumferentially disposed wires comprises an elastic material.

5. The contraceptive device of claim 1, wherein the securing portion is biased to assume the radially expanded state.

6. The contraceptive device of claim 1, wherein the securing portion is configured to assume the unexpanded state when a force is applied to an outer surface of the securing portion.

7. The contraceptive device of claim 6, wherein the securing portion is configured to assume the expanded state when the force is removed.

8. The contraceptive device of claim 1, further comprising a marker disposed about an outer surface of the contraceptive device, wherein the marker is configured for visualization using a visualization device.

9. The contraceptive device of claim 1, wherein the retrieval member comprises a hook, loop, or a magnet.

10. A method for delivering a fallopian tube filter comprising:
providing a fallopian tube filter comprising a retrieval member wherein the retrieval member comprises an engagement portion configured to engage a grasping member for the removal of the fallopian tube filter, wherein the fallopian tube filter is configured for reversibly sealing a passageway from sperm and eggs and the fallopian tube filter is configured to engage and penetrate tissue and resist tissue in-growth thereabout to permit removal of an entirety of the fallopian tube filter with minimal tissue damage;
compressing the fallopian tube filter;
directing the fallopian tube filter to a proximal fallopian tube as deep as the tubal isthmus; and
deploying the fallopian tube filter at the proximal fallopian tube as deep as the tubal isthmus, wherein the retrieval member extends towards a uterus cavity, and wherein the fallopian tube filter expands and applies a radial force to an interior surface of the fallopian tube reversibly sealing the fallopian tube from the passage of sperm and eggs.

11. The method of claim 10, wherein the fallopian tube filter further comprises a marker disposed about an outer surface of the fallopian tube filter, wherein the marker is configured for visualization using a visualization device.

12. The method of claim 12, wherein the visualization device is hysteroscopy, fluoroscopy, x-ray, direct endoscopic visualization, ultrasound, or magnetic resonance imaging (MRI).

13. The method of claim 10, wherein the directing the fallopian tube filter to a proximal fallopian tube as deep as the tubal isthmus further comprises visualizing the fallopian tube filter.

14. The method of claim 10, wherein the retrieval member comprises a hook, loop, or a magnet.

15. The method of claim 10, further comprising removing the fallopian tube filter by engaging the retrieval member.

16. The method of claim 10, wherein, the fallopian tube filter further comprises:
a plurality of longitudinal wires each comprising a body portion and a securing portion disposed within a plane through a longitudinal axis of the fallopian tube filter, wherein the body portion of adjacent wires are connected to each other at a plurality of locations by a plurality of circumferentially disposed wires;
wherein the securing portion of the wires are unconnected and comprise a uniform cross-sectional area and are configured to curve outward from the longitudinal axis;
further wherein the securing portion is configured to engage and penetrate tissue and resist tissue in-growth thereabout to permit removal of an entirety of the fallopian tube filter with minimal tissue damage;
wherein the body portion and the securing portion are configured to move between a radially expanded state and an unexpanded state, wherein an outer diameter of the securing portion when in the radially expanded state is greater than an outer diameter of the securing portion when in the unexpanded state; and
a film in communication with at least a portion of the body portion configured to seal the area between adjacent wires from the passage of sperm and eggs.

17. The method of claim 16, wherein body portion comprises a proximal section and a distal section disposed distally of the proximal section, wherein a diameter of the body portion increases distally along the longitudinal axis, and the diameter of the distal section increases at a faster rate than the diameter of the proximal section

18. An occlusion medical device comprising:
a conical configuration of a plurality of atraumatic wires comprising a body portion and a securing portion, wherein the securing portion is configured to move between a radially expanded state and an unexpanded state, wherein an outer diameter of the securing portion when in the radially expanded state is greater than the outer diameter of the securing portion when in the unexpanded state, further wherein the securing portion is configured to engage and penetrate tissue and resist tissue in-growth thereabout to permit removal of an entirety of the medical device with minimal tissue damage;
a retrieval member connected to the body portion; and
a sealing member in communication with at least a portion of the body portion to seal the medical device from the passage of a fluid and a solid therethrough.

19. The occlusion medical device of claim 18, wherein the sealing member comprises Thoralon.

20. The occlusion medical device of claim 18, wherein the retrieval member comprises a hook, loop, or a magnet.
